(19) United States
(12) Patent Application Publication
(10) Pub. No.: US 2009/0318886 A1
(43) Pub. Date: Dec. 24, 2009

(54) MEDICAL DEVICE

(76) Inventor: Anna Norlin, Stockholm (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/298,164

(22) PCT Filed: Apr. 27, 2006
(86) PCT No.: PCT/SE2006/000514

§ 371 Date: Mar. 25, 2009

(51) Int. Cl. A61N 1/05; A61M 37/00; A61M 25/09

(52) U.S. Cl. 604/500; 607/120; 604/57

(57) ABSTRACT

An implantable medical device can be configured to directly or indirectly release nitric oxide during its insertion in a patient. Such a device can reduce the amount of time required for implantation as well as reduce or prevent damage to the region surrounding the implanted device.

### BACKGROUND OF THE INVENTION

1. Field of the Invention

The present invention relates generally to human and veterinary medical devices and to methods for their fabrication and use. More particularly, it relates to insertable or implantable medical devices configured to release a material, for example, by having a dispersible coating. In particular, the invention relates to implantable cardiac leads and devices designed to facilitate implantation thereof, which incorporate a direct or indirect nitric oxide releaser.

2. Description of the Prior Art

Advances in medicine provide physicians and health care workers with ever-improving tools with which to treat patients and prevent illness. Devices which are partially or completely inserted in a patient temporarily or on an extended basis are at times the preferred way to effect a particular treatment. Insertions of devices in the vascular system are common, for example, a cardiac pacemaker can be implanted in the chest of a patient, the pacemaker being connected to electrodes which extend into the patient's heart. Other examples include the insertion of stents, catheters, balloons, or objects such as guidewires or stylets which can facilitate the insertion of any number of devices.

When implanting a pacemaker, the number of electrodes and where in the heart they are located depends on the particular therapeutical approach and equipment used. Two electrical leads can be fed through the venous system, one fixed in the wall of the right atrium, the other fixed in the wall of the right ventricle. In another procedure, three leads extend from the pacemaker through the venous system and enter the right atrium where one lead is fixed, the second fixed in the right ventricle and the third is fed through the coronary sinus to a cardiac vein on the left ventricle. This configuration allows for cardiac resynchronization therapy but presents greater challenges for the treating physician since the left ventricle lead has a convoluted shape and proper placement requires that the lead is carefully guided through a maze of narrow venous passageways, which differ from patient to patient due to anatomical variations. The vessel walls can have preexisting damage; introduction of the device and its manipulation through the vascular system can further cause undesirable injury to the adjacent vessel walls.

Research to date has provided some tools which facilitate electrode leads. Materials and methods are constantly improving and making these elements smaller, more flexible, and more biocompatible. Multi-lumen devices are smaller than their predecessors, and coatings that help the device glide can be applied on the outer surface. The thin, pliable, glidable leads are easier to thread through the vascular system and can reduce the risk of injury during insertion. Systemic administration of, e.g., anti-thrombotic therapies can help reduce the risk of injury to the patient during insertion. However, further developments are required to help make the insertion process quicker, easier, and safer.

Focusing on the long term resideability of implantable devices, researchers have noted there are problems with clot formation. One solution has been to provide a device which slowly releases nitric oxide over its lifetime. Nitric oxide (NO) inhibits platelet adhesion and activation and thus helps prevent clots from forming.

Thus, where long-term biocompatibility is desired, one can increase the hemocompatibility of devices by, for example, coating implants with a compound which reacts with protein-bound NO in the bloodstream to release NO and thus decrease or prevent platelet adhesion (see Gappa-Fahlenkamp and Lewis, Improved hemocompatibility of poly(ethylene terephthalate) modified with various thiol-containing groups, Biomaterials 26 (2005) 3479-3485 and Frost et al., Polymers incorporating NO releasing/generating substances for improved biocompatibility of blood-contacting medical devices, Biomaterials 26 (2005) 1685-1693).

Additional research into anti-microbial coatings revealed that implants coated with a NO-containing film cause local oxidative stress during NO release, thus acting in an anti-microbial fashion (see Nablo et al., Inhibition of implant-associated infections via NO release, Biomaterials 26 (2005) 6984-6990).

While this technology may be helpful for the long-term biocompatibility of devices, it leaves unspecified and unaddressed the problem of the time-consuming and arduous insertion of leads, particularly left ventricle leads. Similar issues can also arise with the insertion of other devices, whether these devices are intended for short-term use or permanent indwelling in a patient. Thus, there remains a need in the art to address the unique problems of insertion with novel solutions. The present invention fulfils this and other needs, and addresses other deficiencies of prior art implementations and techniques.

### SUMMARY OF THE INVENTION

An object of the present invention is to provide a device which is more easily inserted or implanted into a patient and methods for inserting or implanting such a device. This object is achieved by a device which releases NO during implantation. The device can directly or indirectly release NO. Examples of amounts of NO which can be released during insertion are 0.1-1000 picomol/cm², which can allow for a minimal amount of additional coating or configuration of the device but still accomplish a release of NO during insertion or implantation, up to a number of hours.

As used herein, “insert,” “insertable,” “implant,” and “implantable” apply equally to devices which are intended for transient use as well as devices which are intended for long-term implantation.

The distal end of the device of the present invention may comprise at least one opening through which a substance which directly or indirectly releases nitric oxide is discharged. Alternately, the distal end may be coated with an appropriated NO-releasing material. NO may be discharged in a pulsing fashion, at intervals equivalent to systolic intervals of a patient hosting the device.

According to an embodiment of the invention, the device is a guidewire or stylet. Such a device can continually release NO while in contact with a patient.

According to a further embodiment of the invention, the device is a cardiac pacemaker electrode. Such an electrode is configured to release NO during implantation, which can take about one or about two hours in simpler cases. In more complex cases, examples can be about three hours, about four hours, or about five hours. One way to configure such an electrode is to provide NO release in such a way that, for example, 50%, 75%, 90%, or 95% of the total NO release from the device occurs during the implantation.

According to a further embodiment of the invention, a method of implanting a device in a patient is provided which comprises providing an elongate implantable device having a distal end and directly or indirectly releasing nitric oxide from the distal end during implantation. The device can be implanted into a vessel of the patient. The method can be configured to increase blood flow in the vessel, and/or dilate the vessel.

According to a further embodiment of the invention, an implantable device having an elongated portion with a distal end is provided, along with means for directly or indirectly releasing nitric oxide during implantation of the device in a patient provided on or in said distal end.

### DESCRIPTION OF THE PREFERRED EMBODIMENTS

As noted above, the invention is concerned with devices which release NO during insertion or implantation. The present invention is applicable to mammals, such as humans and domestic or research animals or livestock. For convenience, the recipient of the device is described herein as the patient.

The natural release of intravascular NO occurs when the endothelial cells lining the vessels release a puff of NO at each systole. This diffuses into the underlying smooth muscle cells, causing them to relax and permitting a surge of blood to pass. NO also inhibits the aggregation of platelets and prevents clotting from interfering with blood flow. Further, NO inhibits inflammation in blood vessels. As it has many physiological functions, the preparation and use of NO-releasing materials has grown widely and at present a number of materials are available for use in the present invention.

According to the invention, NO can be released directly or indirectly. That is, the device itself can directly release NO by, for example, a stream of NO which is passed through the device to its distal end. This can be accomplished by providing a device with a lumen through which NO can pass from an external source to an opening at the distal end of the device. Alternatively, a coating containing NO in actively-releasable form can be applied to the device, either during manufacture or immediately prior to use, or both. One example is to provide NO in a sugar solution which is rapidly dissolved once in contact with patient fluids, thereby releasing the NO. Existing devices could be dipped in or otherwise treated with this solution prior to insertion to take advantage of the benefits of the present invention and facilitate insertion of the device.

Alternatively, the NO can be indirectly released, for example, by stimulating nearby endothelial cells to release NO. Indirect release can rely on the patient's own cells since NO is found in nearly every cell type, where it is enzymatically synthesized through the oxidization of L-arginine to N-omega-hydroxy-L-arginine, which is converted into L-citrulline and an uncharged NO free radical. Three enzymes, neuronal, endothelial, and inducible NO synthase, are involved in the process. Using techniques and materials known to the skilled worker, appropriate materials can be present on or in the device which affect this pathway and/or otherwise cause NO present in cells to be released.

Indirect release of NO can also occur by releasing compounds which, once in contact with a patient's blood or bodily fluids, convert to NO. The selection of the appropriate precursor compound depends upon which tissues and fluids the device will be expected to contact during insertion, such precursors are known in the art. For example, nitroglycerine can be used in a coating or released from the distal end of a device; it is readily available in numerous formulations such as ointments and solutions and rapidly degrades in the mammalian body to NO.

The present device and methods related thereto are effective even in cases where insertion causes a device to move against blood flow or counter current to the movement of patient bodily fluids. For example, when implanting a cardiac pacemaker lead, the distal end of the lead travels into the heart while the blood passing the distal end progresses back along the length of the lead. NO released at the distal end nonetheless causes dilation of the vessel located near the tip of the device, as well as in a direction along the length of the lead, thus resulting in the beneficial effect. In some applications, a device which requires substantial dilation or rapid blood flow can be preceded during insertion by a distal end which tapers to a narrow tip. This narrow tip can optionally be dissolvable. Rapidly dissipated NO from the narrow distal end can thereby facilitate insertion of the larger, wider device.

When the distal end of the device is in the vascular system and is releasing NO, it will also have the beneficial side effect of increasing blood flow. This helps prevent complications from clotting and thrombosis. Systemic administration of anti-thrombotic agents may thus be avoided, potentially reducing the overall cost of materials and also reducing stress on the patient's system during certain applications.

Thus, instead of relying on teachings that NO loss during implantation should be minimized—so as to prolong the duration of NO release during the lifetime of the device—the present invention provides a device configured to release NO during implantation in an effort to make the insertion procedure quicker, easier, and safer.

### EXAMPLE 1

Indirect Release with a Coated Device.

A NO-generating device is prepared by first providing an implantable device comprising a biocompatible material. L-cysteine or an L-cystiene-containing compound is immobilized to the surface of the device. When the device is inserted and contacts the patient's blood plasma, transnitrosation occurs in which NO, which is abundant in the plasma, is transferred from S-nitrosoalbumin to L-cysteine. Because nitrosated L-cysteine is unstable it will then release NO, causing adjacent vascular structures to dilate and facilitating the insertion and placement of the device (see Grappa-Fahlenkamp and Lewis). Such a coated device may be particularly well suited for vascular applications as it relies on components of the patient's blood to release NO. A further benefit of this configuration is the ease with which it can be adapted to existing devices, as the coating may be provided immediately prior to insertion.

### EXAMPLE 2

Direct Release with a Coated Device.

An implantable device is provided, comprising biocompatible materials such as silicon. A sol-gel coating is provided on the device, solidified to a xerogel and modified to comprise NO-donors (see Nablo et al). The coating can be provided on the device at the time or manufacture or prior to use. The degree of NO saturation of the xerogel can be tailored to the chosen application of the device, for example, to release at least 0.1 picomol/cm² and not more than 1000 picomol/cm², for example 100 picomol/cm² of NO for between 1-5 hours, preferably 1-3 hours. If the teaching of Nablo is followed without modification, the resultant device could be primed prior to insertion, so that the initial burst of NO released from the device coincides with the insertion period.

### EXAMPLE 3

Direct Release with a Coated Device.

An implantable device is provided which has a polymeric outer layer or barrier. Examples include polymeric insulation on electrical leads for pacemakers. The polymer is selected based on the potential application of the device, and N-diazeniumdiolate is incorporated into the polymeric structure (see Frost et al). One adjustment to be made to known methods, of course, is to select the appropriate amount and localization of the N-diazeniumdiolate to provide rapid NO release during implantation, instead of conservation and long-term stable release as taught by previous methods.

### EXAMPLE 4

Direct Release through the Device.

A device can be provided, which device has a tubular structure such as a hollow guide wire which can be used to insert “over the wire” cardiac pacemaker electrodes. The hollow tip of the guide wire can be provided with a plurality of openings, or exclusively with the opening at the end of the wire. At the proximal end of the guide wire, located external to the patient during the insertion, a NO source can be connected, such as a fluid or liquid containing NO in rapidly-diffusible form.

Prior to insertion of the guide wire, the flow of NO from the source through the guide wire and out the distal end can be primed. During insertion the flow of NO can be controlled to maximize the beneficial vessel dilation properties as needed. The flow can be terminated prior to removal of the guide wire or after, as determined by the person managing the insertion. In addition to openings at or near the distal end of the guide wire, additional outlets for the NO stream can be provided along the length of the device to help keep vessels in the relaxed state during insertion.

Implantation of devices encompassed by the present invention can be performed as known to those in the art, e.g., implantation of pacemaker leads as known to interventional cardiologists.

Although modifications and changes may be suggested by those skilled in the art, it is the intention of the inventor to embody within the patent warranted heron all changes and modifications as reasonably and properly come within the scope of her contribution to the art.

I claim as my invention—therefore:

1. An implantable device, comprising: a device body having at least one elongate portion having a distal end, wherein said distal end is configured to directly or indirectly release nitric oxide during insertion of the device.

2. A device according to claim 1, wherein said distal end is configured to release nitric oxide at about 0.1-1000 picomol/cm2 during insertion.

3. A device according to claim 1, wherein said distal end comprises at least one opening through which a substance which directly or indirectly releases nitric oxide is discharged.

4. A device according to claim 1, wherein said distal end is configured to discharge said substance at intervals equivalent to systolic intervals of a patient hosting the device.

5. A device according to claim 1, wherein said distal end is coated with a material which directly or indirectly releases nitric oxide.

6. A device according to claim 1, wherein the device body is a guidewire or stylet.

7. A device according to claim 6, wherein said distal end is configured to continually release releases nitric oxide when in contact with a patient.

8. A device according to claim 1, wherein the device body is a cardiac pacemaker electrode.

9. A device according to claim 8, wherein said distal end is configured to release nitric oxide for less than about one hour when in contact with a patient.

10. A device according to claim 8, wherein said distal end is configured to release nitric oxide for less than about five hours when in contact with a patient.

11. A device according to claim 8, wherein said distal end is coated with a material which directly or indirectly releases nitric oxide; and
at least 75% of said coating is released within about five hours of contact with a patient.

12. A method of implanting a device in a patient, comprising:
providing an elongate insertable device having a distal end;
directly or indirectly releasing nitric oxide from said distal end of said device during insertion.

13. A method according to claim 12, comprising inserting said device into a vessel of the patient.

14. A method according to claim 12, comprising directly or indirectly releasing nitric oxide in an amount or at a rate that increases blood flow in said vessel.

15. A method according to claim 12, comprising directly or indirectly releasing nitric oxide in an amount or at a rate that dilates said vessel.

16. A method according to claim 12, comprising releasing said nitric oxide for less than about five hours after a first contact with a patient.

17. (canceled)
